(19) United States
(12) Patent Application Publication
(10) Pub. No.: US 2005/0089886 A1
(43) Pub. Date: Apr. 28, 2005

(54) Novel human phospholipases and polynucleotides encoding the same

(76) Inventors: Yi Hu, The Woodlands, TX (US); Boris Nepomnichy, Houston, TX (US); Gregory Donoho, The Woodlands, TX (US); Erin Hilbun, Houston, TX (US); C. Alexander Turner JR., The Woodlands, TX (US); Alejandro Abuin, The Woodlands, TX (US); Glenn Friedrich, Houston, TX (US); Brian Zambrowicz, The Woodlands, TX (US); Arthur T. Sands, The Woodlands, TX (US)

Correspondence Address: Lance K. Ishimoto;LEXICON GENETICS INCORPORATED, 8800 Technology Forest Place, The Woodlands, TX, 77381, US

(21) Appl. No.: 10/861,920

(22) Filed: Jun. 4, 2004

Related U.S. Application Data
(63) Continuation of application No. 09/804,969, filed on Mar. 13, 2001, now abandoned.
(60) Provisional application No. 60/188,885, filed on Mar. 13, 2000.
(60) Provisional application No. 60/189,693, filed on Mar. 15, 2000.

(51) Int. Cl. C12Q 1/68; C07H 21/04; C12N 9/20

(52) U.S. Cl. 435/6; 435/69.1; 435/198; 435/320.1; 435/325; 536/23.2

(57) ABSTRACT

Novel human polynucleotide and polypeptide sequences are disclosed that can be used in therapeutic, diagnostic, and pharmacogenomic applications.

The present application claims the benefit of U.S. Provisional Application Nos. 60/188,885 and 60/189,693, which were filed on Mar. 13, 2000 and Mar. 15, 2000, respectively, and which are herein incorporated by reference in their entirety.

### 1. INTRODUCTION

The present invention relates to the discovery, identification, and characterization of novel human polynucleotides encoding proteins sharing sequence similarity with mammalian phospholipases. The invention encompasses the described polynucleotides, host cell expression systems, the encoded proteins, fusion proteins, polypeptides and peptides, antibodies to the encoded proteins and peptides, and genetically engineered animals that either lack or over express the disclosed polynucleotides, antagonists and agonists of the proteins, and other compounds that modulate the expression or activity of the proteins encoded by the disclosed polynucleotides that can be used for diagnosis, drug screening, clinical trial monitoring and the treatment of diseases and disorders.

### 2. BACKGROUND OF THE INVENTION

Phospholipases hydrolyze phospholipids and can play a key role in the cell activation and signal transduction. As such, phospholipases have been associated with, inter alia, development, inflammation, infectious disease, and cancer.

### 3. SUMMARY OF THE INVENTION

The present invention relates to the discovery, identification, and characterization of nucleotides that encode novel human proteins, and the corresponding amino acid sequences of these proteins. The novel human proteins (NHPs) described for the first time herein share structural similarity with animal phospholipases, including phospholipase C delta-4.

The novel human nucleic acid (cDNA) sequences described herein encode proteins/open reading frames (ORFs) of 239, 329, 351, 149, 239, 261, 762, 69, and 272 amino acids in length (see SEQ ID NOS: 2, 4, 6, 8,. 10, 12, 15, 17 and 19 respectively).

The invention also encompasses agonists and antagonists of the described NHPs, including small molecules, large molecules, mutant NHPs, or portions thereof, that compete with native NHP, peptides, and antibodies, as well as nucleotide sequences that can be used to inhibit the expression of the described NHPs (e.g., antisense and ribozyme molecules, and gene or regulatory sequence replacement constructs) or to enhance the expression of the described NHP polynucleotides (e.g., expression constructs that place the described polynucleotide under the control of a strong promoter system), and transgenic animals that express a NHP transgene, or “knock-outs” (which can be conditional) that do not express a functional NHP. Knock-out mice can be produced in several ways, one of which involves the use of mouse embryonic stem cells (“ES cells”) lines that contain gene trap mutations in a murine homolog of at least one of the described NHPs. When the unique NHP sequences described in SEQ ID NOS:1-20 are “knocked-out” they provide a method of identifying phenotypic expression of the particular gene as well as a method of assigning function to previously unknown genes. Additionally, the unique NHP sequences described in SEQ ID NOS:1-20 are useful for the identification of coding sequence and the mapping a unique gene to a particular chromosome.

Further, the present invention also relates to processes for identifying compounds that modulate, i.e., act as agonists or antagonists, of NHP expression and/or NHP activity that utilize purified preparations of the described NHPs and/or NHP product, or cells expressing the same. Such compounds can be used as therapeutic agents for the treatment of any of a wide variety of symptoms associated with biological disorders or imbalances.

### 4. DESCRIPTION OF THE SEQUENCE LISTING AND FIGURES

The Sequence Listing provides the sequences of the described NHP ORFs encoding the described NHP amino acid sequences. SEQ ID NO:13 and 20 describe nucleotides encoding a NHP ORF with regions of flanking sequence.

### 5. DETAILED DESCRIPTION OF THE INVENTION

The NHPs, described for the first time herein in SEQ ID NOS: 1-13, are novel proteins that are clearly expressed in, inter alia, human cell lines, human fetal brain, brain, cerebellum, spinal cord, thymus, spleen, testis, thyroid, adrenal gland, small intestine, colon, placenta, adipose, rectum, and gene trapped cells. The described sequences were compiled from gene trapped cDNAs, human genomic sequence, and clones isolated from a human fetal brain cDNA librarys (Edge Biosystems, Gaithersburg, Md.). The NHPs, described for the first time herein in SEQ ID NOS: 14-20, are novel proteins that are clearly expressed in, inter alia, human cell lines, human fetal brain, brain, testis, skeletal muscle, pericardium, trachea, and gene trapped cells. The described sequences were compiled from gene trapped cDNAs, human genomic sequence, and clones isolated from a human trachea CDNA library (Edge Biosystems, Gaithersburg, Md.).

The present invention encompasses the nucleotides presented in the Sequence Listing, host cells expressing such nucleotides, the expression products of such nucleotides, and: (a) nucleotides that encode mammalian homologs of the described polynucleotides, including the specifically described NHPs, and the NHP products; (b) nucleotides that encode one or more portions of the NHPs that correspond to functional domains, and the polypeptide products specified by such nucleotide sequences, including but not limited to the novel regions of any active domain(s); (c) isolated nucleotides that encode mutant versions, engineered or naturally occurring, of the described NHPs in which all or a part of at least one domain is deleted or altered, and the polypeptide products specified by such nucleotide sequences, including but not limited to soluble proteins and peptides in which all or a portion of the signal (or hydrophobic transmembrane) sequence is deleted; (d) nucleotides that encode chimeric fusion proteins containing all or a portion of a coding region of an NHP, or one of its domains ( e.g., a receptor or ligand binding domain, accessory protein/self-association domain, etc.) fused to another peptide or polypeptide; or (e) therapeutic or diagnostic derivatives of the described polynucleotides such as oligonucleotides, antisense polynucleotides, ribozymes, dsRNA, or gene therapy constructs comprising a sequence first disclosed in the Sequence Listing. As discussed above, the present invention includes: (a) the human DNA sequences presented in the Sequence Listing (and vectors comprising the same) and additionally contemplates any nucleotide sequence encoding a contiguous NHP open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product. Additionally contemplated are any nucleotide sequences that hybridize to the complement of a DNA sequence that encodes and expresses an amino acid sequence presented in the Sequence Listing under moderately stringent conditions, e.g., washing in 0.2×SSC/0.1% SDS at 42° C. (Ausubel et al., 1989, supra), yet still encodes a functionally equivalent NHP product. Functional equivalents of a NHP include naturally occurring NHPs present in other species and mutant NHPs whether naturally occurring or engineered (by site directed mutagenesis, gene shuffling, directed evolution as described in, for example, U.S. Pat. No. 5,837,458). The invention also includes degenerate nucleic acid variants of the disclosed NHP polynucleotide sequences.

Additionally contemplated are polynucleotides encoding NHP ORFs, or their functional equivalents, encoded by polynucleotide sequences that are about 99, 95, 90, or about 85 percent similar or identical to corresponding regions of the nucleotide sequences of the Sequence Listing (as measured by BLAST sequence comparison analysis using, for example, the GCG sequence analysis package using standard default settings).

The invention also includes nucleic acid molecules, preferably DNA molecules, that hybridize to, and are therefore the complements of, the described NHP nucleotide sequences. Such hybridization conditions can be highly stringent or less highly stringent, as described above. In instances where the nucleic acid molecules are deoxyoligonucleotides (“DNA oligos”), such molecules are generally about 16 to about 100 bases long, or about 20 to about 80, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such NHP oligonucleotides can be used as hybridization probes for screening libraries, and assessing gene expression patterns (particularly using a micro array or high-throughput “chip” format). Additionally, a series of the described NHP oligonucleotide sequences, or the complements thereof, can be used to represent all or a portion of the described NHP sequences. An oligonucleotide or polynucleotide sequence first disclosed in at least a portion of one or more of the sequences of SEQ ID NOS: 1-20 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS: 1-20, or an amino acid sequence encoded thereby. Methods for attaching biopolymers to, or synthesizing biopolymers on, solid support matrices, and conducting binding studies thereon are disclosed in, inter alia, U.S. Pat. Nos. 5,700,637, 5,556,752, 5,744,305, 4,631,211, 5,445,934, 5,252,743, 4,713,326, 5,424,186, and 4,689,405 the disclosures of which are herein incorporated by reference in their entirety.

Addressable arrays comprising sequences first disclosed in SEQ ID NOS:1-20 can be used to identify and characterize the temporal and tissue specific expression of a gene. These addressable arrays incorporate oligonucleotide sequences of sufficient length to confer the required specificity, yet be within the limitations of the production technology. The length of these probes is within a range of between about 8 to about 2000 nucleotides. Preferably the probes consist of 60 nucleotides and more preferably 25 nucleotides from the sequences first disclosed in SEQ ID NOS:1-20.

For example, a series of the described oligonucleotide sequences, or the complements thereof, can be used in chip format to represent all or a portion of the described sequences. The oligonucleotides, typically between about 16 to about 40 (or any whole number within the stated range) nucleotides in length can partially overlap each other and/or the sequence can be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing and proceed in either a sense (5′-to-3′) orientation vis-a-vis the described sequence or in an antisense orientation.

Microarray-based analysis allows the discovery of broad patterns of genetic activity, providing new understanding of gene functions and generating novel and unexpected insight into transcriptional processes and biological mechanisms. The use of addressable arrays comprising sequences first disclosed in SEQ ID NOS:1-20 provides detailed information about transcriptional changes involved in a specific pathway, potentially leading to the identification of novel components or gene functions that manifest themselves as novel phenotypes.

Probes consisting of sequences first disclosed in SEQ ID NOS:1-20 can also be used in the identification, selection and validation of novel molecular targets for drug discovery. The use of these unique sequences permits the direct confirmation of drug targets and recognition of drug dependent changes in gene expression that are modulated through pathways distinct from the drugs intended target. These unique sequences therefore also have utility in defining and monitoring both drug action and toxicity.

As an example of utility, the sequences first disclosed in SEQ ID NOS:1-20 can be utilized in microarrays or other assay formats, to screen collections of genetic material from patients who have a particular medical condition. These investigations can also be carried out using the sequences first disclosed in SEQ ID NOS:1-20 in silico and by comparing previously collected genetic databases and the disclosed sequences using computer software known to those in the art.

Thus the sequences first disclosed in SEQ ID NOS:1-20 can be used to identify mutations associated with a particular disease and also as a diagnostic or prognostic assay.

Although the presently described sequences have been specifically described using nucleotide sequence, it should be appreciated that each of the sequences can uniquely be described using any of a wide variety of additional structural attributes, or combinations thereof. For example, a given sequence can be described by the net composition of the nucleotides present within a given region of the sequence in conjunction with the presence of one or more specific oligonucleotide sequence(s) first disclosed in the SEQ ID NOS: 1-20. Alternatively, a restriction map specifying the relative positions of restriction endonuclease digestion sites, or various palindromic or other specific oligonucleotide sequences can be used to structurally describe a given sequence. Such restriction maps, which are typically generated by widely available computer programs ( e.g., the University of Wisconsin GCG sequence analysis package, SEQUENCHER 3.0, Gene Codes Corp., Ann Arbor, Mich., etc.), can optionally be used in conjunction with one or more discrete nucleotide sequence(s) present in the sequence that can be described by the relative position of the sequence relatve to one or more additional sequence(s) or one or more restriction sites present in the disclosed sequence.

For oligonucleotide probes, highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as NHP gene antisense molecules, useful, for example, in NHP gene regulation (for and/or as antisense primers in amplification reactions of NHP gene nucleic acid sequences). With respect to NHP gene regulation, such techniques can be used to regulate biological functions. Further, such sequences may be used as part of ribozyme and/or triple helix sequences that are also useful for NHP gene regulation.

Inhibitory antisense or double stranded oligonucleotides can additionally comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide can also comprise at least one modified sugar moiety selected from the group including but not limited to arabinose, 2-fluoroarabinose, xylulose, and hexose.

In yet another embodiment, the antisense oligonucleotide will comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual D-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330). Alternatively, double stranded RNA can be used to disrupt the expression and function of a targeted NHP.

Oligonucleotides of the invention can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988, Nucl Acids Res. 16:3209), and methylphosphonate oligonucleotides can be prepared by use of controlled pore glass polymer supports (Sarin et al., 1988, Proc. Natl. Acad. Sci. U.S.A. 85:7448-7451), etc.

Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual (and periodic updates thereof), Cold Springs Harbor Press, N.Y.; and Ausubel et al., 1989, Current Protocols in Molecular Biology, Green Publishing Associates and Wiley Interscience, N.Y.

Alternatively, suitably labeled NHP nucleotide probes can be used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

Further, a NHP gene homolog can be isolated from nucleic acid from an organism of interest by performing PCR using two degenerate or “wobble” oligonucleotide primer pools designed on the basis of amino acid sequences within the NHP products disclosed herein. The template for the reaction may be total RNA, mRNA, and/or cDNA obtained by reverse transcription of mRNA prepared from human or non-human cell lines or tissue known or suspected to express an allele of a NHP gene.

The PCR product can be subcloned and sequenced to ensure that the amplified sequences represent the sequence of the desired NHP gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

PCR technology can also be used to isolate full length cDNA sequences. For example, RNA can be isolated, following standard procedures, from an appropriate cellular or tissue source (i.e., one known, or suspected, to express a NHP gene). A reverse transcription (RT) reaction can be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment can be isolated. For a review of cloning strategies that can be used, see e.g., Sambrook et al., 1989, supra.

A cDNA encoding a mutant NHP gene can be isolated, for example, by using PCR. In this case, the first cDNA strand may be synthesized by hybridizing an oligo-dT oligonucleotide to mRNA isolated from tissue known or suspected to be expressed in an-individual putatively carrying a mutant NHP allele, and by extending the new strand with reverse transcriptase. The second strand of the cDNA is then synthesized using an oligonucleotide that hybridizes specifically to the 5′ end of the normal gene. Using these two primers, the product is then amplified via PCR, optionally cloned into a suitable vector, and subjected to DNA sequence analysis through methods well known to those of skill in the art. By comparing the DNA sequence of the mutant NHP allele to that of a corresponding normal NHP allele, the mutation(s) responsible for the loss or alteration of function of the mutant NHP gene product can be ascertained.

Alternatively, a genomic library can be constructed using DNA obtained from an individual suspected of or known to carry a mutant NHP allele (e.g., a person manifesting a NHP-associated phenotype such as, for example, obesity, high blood pressure, connective tissue disorders, infertility, etc.), or a cDNA library can be constructed using RNA from a tissue known, or suspected, to express a mutant NHP allele. A normal NHP gene, or any suitable fragment thereof, can then be labeled and used as a probe to identify the corresponding mutant NHP allele in such libraries. Clones containing mutant NHP gene sequences can then be purified and subjected to sequence analysis according to methods well known to those skilled in the art.

Additionally, an expression library can be constructed utilizing cDNA synthesized from, for example, RNA isolated from a tissue known, or suspected, to express a mutant NHP allele in an individual suspected of or known to carry such a mutant allele. In this manner, gene products made by the putatively mutant tissue can be expressed and screened using standard antibody screening techniques in conjunction with antibodies raised against a normal NHP product, as described below. (For screening techniques, see, for example, Harlow, E. and Lane, eds., 1988, “Antibodies: A Laboratory Manual”, Cold Spring Harbor Press, Cold Spring Harbor, N.Y.).

Additionally, screening can be accomplished by screening with labeled NHP fusion proteins, such as, for example, alkaline phosphatase-NHP or NHP-alkaline phosphatase fusion proteins. In cases where a NHP mutation results in an expressed gene product with altered function (e.g., as a result of a missense or a frameshift mutation), polyclonal antibodies to a NHP are likely to cross-react with a corresponding mutant NHP gene product. Library clones detected via their reaction with such labeled antibodies can be purified and subjected to sequence analysis according to methods well known in the art.

The invention also encompasses (a) DNA vectors that contain any of the foregoing NHP coding sequences and/or their complements (i.e., antisense); (b) DNA expression vectors that contain any of the foregoing NHP coding sequences operatively associated with a regulatory element that directs the expression of the coding sequences (for example, baculo virus as described in U.S. Pat. No. 5,869,336 herein incorporated by reference); (c) genetically engineered host cells that contain any of the foregoing NHP coding sequences operatively associated with a regulatory element that directs the expression of the coding sequences in the host cell; and (d) genetically engineered host cells that express an endogenous NHP gene under the control of an exogenously introduced regulatory element (i.e., gene activation). As used herein, regulatory elements include, but are not limited to, inducible and non-inducible promoters, enhancers, operators and other elements known to those skilled in the art that drive and regulate expression. Such regulatory elements include but are not limited to the cytomegalovirus (hCMV) immediate early gene, regulatable, viral elements (particularly retroviral LTR promoters), the early or late promoters of SV40 adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

The present invention-also encompasses antibodies and anti-idiotypic antibodies (including Fab fragments), antagonists and agonists of a NHP, as well as compounds or nucleotide constructs that inhibit expression of a NHP gene (transcription factor inhibitors, antisense and ribozyme molecules, or gene or regulatory sequence replacement constructs), or promote the expression of a NHP ( e.g., expression constructs in which NHP coding sequences are operatively associated with expression control elements such as promoters, promoter/enhancers, etc.).

The NHPs or NHP peptides, NHP fusion proteins, NHP nucleotide sequences, antibodies, antagonists and agonists can be useful for the detection of mutant NHPs or inappropriately expressed NHPs for the diagnosis of disease. The NHP proteins or peptides, NHP fusion proteins, NHP nucleotide sequences, host cell expression systems, antibodies, antagonists, agonists and genetically engineered cells and animals can be used for screening for drugs (or high throughput screening of combinatorial libraries) effective in the treatment of the symptomatic or phenotypic manifestations of perturbing the normal function of NHP in the body. The use of engineered host cells and/or animals may offer an advantage in that such systems allow not only for the identification of compounds that bind to the endogenous receptor for an NHP, but can also identify compounds that trigger NHP-mediated activities or pathways.

Finally, the NHP products can be used as therapeutics. For example, soluble derivatives such as NHP peptides/domains corresponding to NHPs, NHP fusion protein products (especially NHP-Ig fusion proteins, i.e., fusions of a NHP, or a domain of a NHP, to an IgFc), NHP antibodies and anti-idiotypic antibodies (including Fab fragments), antagonists or agonists (including compounds that modulate or act on downstream targets in a NHP-mediated pathway) can be used to directly treat diseases or disorders. For instance, the administration of an effective amount of soluble NHP, or a NHP-IgFc fusion protein or-an anti-idiotypic antibody (or its Fab) that mimics the NHP could activate or effectively antagonize the endogenous NHP receptor. Nucleotide constructs encoding such NHP products can be used to genetically engineer host cells to express such products in vivo; these genetically engineered cells function as “bioreactors” in the body delivering a continuous supply of a NHP, a NHP peptide, or a NHP fusion protein to the body. Nucleotide constructs encoding functional NHPs, mutant NHPs, as well as antisense and ribozyme molecules can also be used in “gene therapy” approaches for the modulation of NHP expression. Thus, the invention also encompasses pharmaceutical formulations and methods for treating biological disorders.

Various aspects of the invention are described in greater detail in the subsections below.

### 5.1 THE NHP SEQUENCES

The cDNA sequences and the corresponding deduced amino acid sequences of the described NHPs are presented in the Sequence Listing. SEQ ID NOS:13 and 20 describe NHP ORFs as well as flanking regions. The NHP nucleotides were obtained from human cDNA libraries using probes and/or primers generated from human gene trapped sequence tags. Expression analysis has provided evidence that some of the described NHPs are widely expressed (SEQ ID NOS:1-13) and some of the described NHPs (SEQ ID NOS:14-20) have a fairly restricted pattern of expression including those that share structural similarity with phospholipase C delta-4 . Given the importance of phospholipases, similar molecules and activities have been subject to considerable scientific scrutiny as demonstrated in U.S. Pat. Nos.5,859,222 and 5,587,306, both of which are herein incorporated by reference in their entirety, which describe molecules encoding phospholipase activities that are similar to those of the disclosed NHPs as well as a variety of uses and applications for which the described NHPs can be applied.

### 5.2 NHPS AND NHP POLYPEPTIDES

NHPs, polypeptides, peptide fragments, mutated, truncated, or deleted forms of the NHPs, and/or NHP fusion proteins can be prepared for a variety of uses. These uses include but are not limited to the generation of antibodies, as reagents in diagnostic assays, the identification of other cellular gene products related to a NHP, as reagents in assays for screening for compounds that can be as pharmaceutical reagents useful in the therapeutic treatment of mental, biological, or medical disorders and diseases. Given the similarity information and expression data, the described NHPs can be targeted (by drugs, oligos, antibodies, etc,) in order to treat disease, or to therapeutically augment the efficacy of, for example, chemotherapeutic agents used in the treatment of breast or prostate cancer.

The Sequence Listing discloses the amino acid sequences encoded by the described NHP polynucleotides. The NHPs typically display have initiator methionines in DNA sequence contexts consistent with a translation initiation site.

The NHP amino acid sequences of the invention include the amino acid sequence presented in the Sequence Listing as well as analogues and derivatives thereof. Further, corresponding NHP homologues from other species are encompassed by the invention. In fact, any NHP protein encoded by the NHP nucleotide sequences described above are within the scope of the invention, as are any novel polynucleotide sequences encoding all or any novel portion of an amino acid sequence presented in the Sequence Listing. The degenerate nature of the genetic code is well known, and, accordingly, each amino acid presented in the Sequence Listing, is generically representative of the well known nucleic acid “triplet” codon, or in many cases codons, that can encode the amino acid. As such, as contemplated herein, the amino acid sequences presented in the Sequence Listing, when taken together with the genetic code (see, for example, Table 4-1 at page 109 of “Molecular Cell Biology”, 1986, J. Darnell et al. eds., Scientific American Books, New York, N.Y., herein incorporated by reference) are generically representative of all the various permutations and combinations of nucleic acid sequences that can encode such amino acid sequences.

The invention also encompasses proteins that are functionally equivalent to the NHPs encoded by the presently described nucleotide sequences as judged by any of a number of criteria, including, but not limited to, the ability to bind and cleave a substrate of a NHP, or the ability to effect an identical or complementary downstream pathway, or a change in cellular metabolism (e.g., proteolytic activity, ion flux, tyrosine phosphorylation, etc.). Such functionally equivalent NHP proteins include, but are not limited to, additions or substitutions of amino acid residues within the amino acid sequence encoded by the NHP nucleotide sequences described above, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

A variety of host-expression vector systems can be used to express the NHP nucleotide sequences of the invention. Where, as in the present instance, the NHP peptide or polypeptide is thought to be membrane protein, the hydrophobic regions of the protein can be excised and the resulting soluble peptide or polypeptide can be recovered from the culture media. Such expression systems also encompass engineered host cells that express a NHP, or functional equivalent, in situ. Purification or enrichment of a NHP from such expression systems can be accomplished using appropriate detergents and lipid micelles and methods well known to those skilled in the art. However, such engineered host cells themselves may be used in situations where it is important not only to retain the structural and functional characteristics of the NHP, but to assess biological activity, e.g., in drug screening assays.

The expression systems that may be used for purposes of the invention include but are not limited to microorganisms such as bacteria (e.g., E. coli, B. subtilis) transformed with recombinant bacteriophage DNA, plasmid DNA or cosmid DNA expression vectors containing NAP nucleotide sequences; yeast (e.g., Saccharomiyces, Pichia) transformed with recombinant yeast expression vectors containing NHP nucleotide sequences; insect cell systems infected with recombinant virus expression vectors (e.g., baculovirus) containing NHP sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing NHP nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the NHP product being expressed. For example, when a large quantity of such a protein is to be produced for the generation of pharmaceutical compositions of or containing NHP, or for raising antibodies to a NHP, vectors that direct the expression of high levels of fusion protein products that are readily purified may be desirable. Such vectors include, but are not limited, to the E. coli expression vector pUR278 (Ruther et al., 1983, EMBO J. 2:1791), in which a NHP coding sequence may be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509)-; and the like. pGEX vectors (Pharmacia or American Type Culture Collection) can also be used to express foreign polypeptides as fusion proteins with glutathione S-transferase (GST). In general, such fusion proteins are soluble and can easily be purified from lysed cells by adsorption to glutathione-agarose-beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. A NHP coding sequence may be cloned individually into non-essential regions (for example the polyhedrin gene) of the virus and placed under control of an AcNPV promoter (for example the polyhedrin promoter). Successful insertion of NHP coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (e.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, the NHP nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing a NHP product in infected hosts (e.g., See Logan & Shenk, 1984, Proc. Natl. Acad. Sci. USA 81:3655-3659). Specific initiation signals may also be required for efficient translation of inserted NHP nucleotide sequences. These signals include the ATG initiation codon and adjacent sequences. In cases where an entire NHP gene or cDNA, including its own initiation codon and adjacent sequences, is inserted into the appropriate expression vector, no additional translational control signals may be needed. However, in cases where only a portion of a NHP coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bitter et al., 1987, Methods in Enzymol. 153:516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines which stably express the NHP sequences described above can be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the NHP product. Such engineered cell lines may be particularly useful in screening and evaluation of compounds that affect the endogenous activity of the NHP product.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, any fusion protein can be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88:8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

Also encompassed by the present invention are fusion proteins that direct the NHP to a target organ and/or facilitate transport across the membrane into the cytosol. Conjugation of NHPs to antibody molecules or their Fab fragments could be used to target cells bearing a particular epitope. Attaching the appropriate signal sequence to the NHP would also transport the NHP to the desired location within the cell. Alternatively targeting of NHP or its nucleic acid sequence might be achieved using liposome or lipid complex based delivery systems. Such technologies are described in Liposomes:A Practical Approach, New, RRC ed., Oxford University Press, New York and in U.S. Pat. Nos. 4,594,595, 5,459,127, 5,948,767 and 6,110,490 and their respective disclosures which are herein incorporated by reference in their entirety. Additionally embodied are novel protein constructs engineered in such a way that they facilitate transport of the NHP to the target site or desired organ. This goal may be achieved by coupling of the NHP to a cytokine or other ligand that provides targeting specificity, and/or to a protein transducing domain (see generally U.S. applications Ser. No. 60/111,701 and 60/056,713, both of which are herein incorporated by reference, for examples of such transducing sequences) to facilitate passage across cellular membranes if needed and can optionally be engineered to include nuclear localization sequences when desired.

### 5.3 ANTIBODIES TO NHP PRODUCTS

Antibodies that specifically recognize one or more epitopes of a NHP, or epitopes of conserved variants of a NHP, or peptide fragments of a NHP are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of NHP in a biological sample and may, therefore, be utilized as part of a diagnostic or prognostic technique whereby patients may be tested for abnormal amounts of NHP. Such antibodies may also be utilized in conjunction with, for example, compound screening schemes for the evaluation of the effect of test compounds on expression and/or activity of a NHP gene product. Additionally, such antibodies can be used in conjunction gene therapy to, for example, evaluate the normal and/or engineered NHP-expressing cells prior to their introduction into the patient. Such antibodies may additionally be used as a method for the inhibition of abnormal NHP activity. Thus, such antibodies may, therefore, be utilized as part of treatment methods.

For the production of antibodies, various host animals may be immunized by injection with a NHP, an NHP peptide (e.g., one corresponding to a functional domain of an NHP), truncated NHP polypeptides (NHP in which one or more domains have been deleted), functional equivalents of the NHP or mutated variant of the NHP. Such host animals may include but are not limited to pigs, rabbits, mice, goats, and rats, to name but a few. Various adjuvants may be used to increase the immunological response, depending on the host species, including but not limited to Freund's adjuvant (complete and incomplete), mineral salts such as aluminum hydroxide or aluminum phosphate, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and or coupling with molecules such as keyhole limpet hemocyanin, tetanus toxoid, diptheria toxoid, ovalbumin, cholera toxin or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique which provides for the production of antibody molecules by continuous cell lines in culture. These include, but are not limited to, the hybridoma technique of Kohler and Milsteini (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,075,181 and 5,877,397 and their respective disclosures which are herein incorporated by reference in their entirety. Also encompassed by the present invention is the use of fully humanized monoclonal antibodies as described in U.S. Pat. No. 6,150,584 and respective disclosures which are herein incorporated by reference in their entirety.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 341:544-546) can be adapted to produce single chain antibodies against NHP gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include, but are not limited to: the F(ab′)₂ fragments which may be produced by pepsin digestion of the antibody molecule and the Fab fragments which may be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Antibodies to a NHP can, in turn, be utilized to generate anti-idiotype antibodies that “mimic” a given NHP, using techniques well known to those skilled in the art. (See, e.g., Greenspan & Bona, 1993, FASEB J 7(5):437-444; and Nissinoff, 1991, J. Immunol. 147(8):2429-2438). For example antibodies which bind to a NHP domain and competitively inhibit the binding of NHP to its cognate receptor can be used to generate anti-idiotypes that “mimic” the NHP and, therefore, bind and activate or neutralize a receptor. Such anti-idiotypic antibodies or Fab fragments of such anti-idiotypes can be used in therapeutic regimens involving a NHP mediated pathway.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims. All cited publications, patents, and patent applications are herein incorporated by reference in their entirety.

```
# SEQUENCE LIS
#TING
<160> NUMBER OF SEQ ID NOS: 20
<210> SEQ ID NO 1
<211> LENGTH: 720
<212> TYPE: DNA
<213> ORGANISM: homo sapiens
<400> SEQUENCE: 1
atgctagcag ttaggaaggc caggaggaaa ctcaggatgg ggaccatctg ct
#cccccaac 60
cccagcggga caaagacatc atcggaggtc tgcaatgccg actggatggc ct
#cgctcccc 120
cctcacctcc acaacctccc cctttccaat ctggcaatcc caggctcaca tg
#attcattc 180
agctactggg tggatgaaaa gtccccagtg gggcctgacc aaacccaagc ta
#tcaaacgc 240
ctcgccagga tctccttggt gaagaagcta atgaagaagt ggtctgtgac tc
#agaacctg 300
acatttcgag aacagctgga agctgggatc cgctactttg acctgcgtgt gt
#cttccaaa 360
ccaggggatg ccgaccagga gatctacttc atccatgggc tttttggcat ca
#aggtctgg 420
gatgggctga tggaaattga ctcgtttctt acacagcacc cccaggagat ta
#tcttcctg 480
gatttcaacc acttctatgc catggatgag acccatcaca aatgcctggt tc
#tgcggatc 540
caggaggcct ttggaaacaa gctgtgccca gcctgcagtg tggaaagttt ga
#cgctgcga 600
actctgtggg agaagaactg ccaggtagga gagataaact tccaagagca ag
#aatttaac 660
tcttctgctt ttcctgtatt gccggctgta aaatcactca atccagggct ct
#taggctaa 720
<210> SEQ ID NO 2
<211> LENGTH: 239
<212> TYPE: PRT
<213> ORGANISM: homo sapiens
<400> SEQUENCE: 2
Met Leu Ala Val Arg Lys Ala Arg Arg Lys Le
#u Arg Met Gly Thr Ile
  1 5
# 10
# 15
Cys Ser Pro Asn Pro Ser Gly Thr Lys Thr Se
#r Ser Glu Val Cys Asn
                        20
# 25
# 30
Ala Asp Trp Met Ala Ser Leu Pro Pro His Le
#u His Asn Leu Pro Leu
                35
# 40
# 45
Ser Asn Leu Ala Ile Pro Gly Ser His Asp Se
#r Phe Ser Tyr Trp Val
        50
# 55
# 60
Asp Glu Lys Ser Pro Val Gly Pro Asp Gln Th
#r Gln Ala Ile Lys Arg
65
#70
#75
#80
Leu Ala Arg Ile Ser Leu Val Lys Lys Leu Me
#t Lys Lys Trp Ser Val
                                85
# 90
# 95
Thr Gln Asn Leu Thr Phe Arg Glu Gln Leu Gl
#u Ala Gly Ile Arg Tyr
                        100
# 105
# 110
Phe Asp Leu Arg Val Ser Ser Lys Pro Gly As
#p Ala Asp Gln Glu Ile
                115
# 120
# 125
Tyr Phe Ile His Gly Leu Phe Gly Ile Lys Va
#l Trp Asp Gly Leu Met
        130
# 135
# 140
Glu Ile Asp Ser Phe Leu Thr Gln His Pro Gl
#n Glu Ile Ile Phe Leu
145 1
#50 1
#55 1
#60
Asp Phe Asn His Phe Tyr Ala Met Asp Glu Th
#r His His Lys Cys Leu
                                165
# 170
# 175
Val Leu Arg Ile Gln Glu Ala Phe Gly Asn Ly
#s Leu Cys Pro Ala Cys
                        180
# 185
# 190
Ser Val Glu Ser Leu Thr Leu Arg Thr Leu Tr
#p Glu Lys Asn Cys Gln
                195
# 200
# 205
Val Gly Glu Ile Asn Phe Gln Glu Gln Glu Ph
#e Asn Ser Ser Ala Phe
        210
# 215
# 220
Pro Val Leu Pro Ala Val Lys Ser Leu Asn Pr
#o Gly Leu Leu Gly
225 2
#30 2
#35
<210> SEQ ID NO 3
<211> LENGTH: 990
<212> TYPE: DNA
<213> ORGANISM: homo sapiens
<400> SEQUENCE: 3
atgctagcag ttaggaaggc caggaggaaa ctcaggatgg ggaccatctg ct
#cccccaac 60
cccagcggga caaagacatc atcggaggtc tgcaatgccg actggatggc ct
#cgctcccc 120
cctcacctcc acaacctccc cctttccaat ctggcaatcc caggctcaca tg
#attcattc 180
agctactggg tggatgaaaa gtccccagtg gggcctgacc aaacccaagc ta
#tcaaacgc 240
ctcgccagga tctccttggt gaagaagcta atgaagaagt ggtctgtgac tc
#agaacctg 300
acatttcgag aacagctgga agctgggatc cgctactttg acctgcgtgt gt
#cttccaaa 360
ccaggggatg ccgaccagga gatctacttc atccatgggc tttttggcat ca
#aggtctgg 420
gatgggctga tggaaattga ctcgtttctt acacagcacc cccaggagat ta
#tcttcctg 480
gatttcaacc acttctatgc catggatgag acccatcaca aatgcctggt tc
#tgcggatc 540
caggaggcct ttggaaacaa gctgtgccca gcctgcagtg tggaaagttt ga
#cgctgcga 600
actctgtggg agaagaactg ccaggttctt attttctacc actgtccctt ct
#acaagcag 660
taccccttcc tgtggccagg aaagaagatt ccagcgccct gggcaaacac ca
#caagtgtg 720
cgcaaactaa tcctcttctt ggagaccact ctgagtgagc gggcctcacg gg
#gctccttc 780
catgtctccc aagcgatcct cacccccaga gtgaagacca ttgcccgggg ct
#tggttggg 840
ggcctcaaga acacgctggt tcataggaat cttcctgcca tcctggactg gg
#tgaaaact 900
cagaagcctg gagccatggg tgtcaacatc atcacatctg acttcgtgga cc
#tggtggac 960
tttgctgcga ctgtcatcaa agttgaatga
#
# 990
<210> SEQ ID NO 4
<211> LENGTH: 329
<212> TYPE: PRT
<213> ORGANISM: homo sapiens
<400> SEQUENCE: 4
Met Leu Ala Val Arg Lys Ala Arg Arg Lys Le
#u Arg Met Gly Thr Ile
  1 5
# 10
# 15
Cys Ser Pro Asn Pro Ser Gly Thr Lys Thr Se
#r Ser Glu Val Cys Asn
                        20
# 25
# 30
Ala Asp Trp Met Ala Ser Leu Pro Pro His Le
#u His Asn Leu Pro Leu
                35
# 40
# 45
Ser Asn Leu Ala Ile Pro Gly Ser His Asp Se
#r Phe Ser Tyr Trp Val
        50
# 55
# 60
Asp Glu Lys Ser Pro Val Gly Pro Asp Gln Th
#r Gln Ala Ile Lys Arg
65
#70
#75
#80
Leu Ala Arg Ile Ser Leu Val Lys Lys Leu Me
#t Lys Lys Trp Ser Val
                                85
# 90
# 95
Thr Gln Asn Leu Thr Phe Arg Glu Gln Leu Gl
#u Ala Gly Ile Arg Tyr
                        100
# 105
# 110
Phe Asp Leu Arg Val Ser Ser Lys Pro Gly As
#p Ala Asp Gln Glu Ile
                115
# 120
# 125
Tyr Phe Ile His Gly Leu Phe Gly Ile Lys Va
#l Trp Asp Gly Leu Met
        130
# 135
# 140
Glu Ile Asp Ser Phe Leu Thr Gln His Pro Gl
#n Glu Ile Ile Phe Leu
145 1
#50 1
#55 1
#60
Asp Phe Asn His Phe Tyr Ala Met Asp Glu Th
#r His His Lys Cys Leu
                                165
# 170
# 175
Val Leu Arg Ile Gln Glu Ala Phe Gly Asn Ly
#s Leu Cys Pro Ala Cys
                        180
# 185
# 190
Ser Val Glu Ser Leu Thr Leu Arg Thr Leu Tr
#p Glu Lys Asn Cys Gln
                195
# 200
# 205
Val Leu Ile Phe Tyr His Cys Pro Phe Tyr Ly
#s Gln Tyr Pro Phe Leu
        210
# 215
# 220
Trp Pro Gly Lys Lys Ile Pro Ala Pro Trp Al
#a Asn Thr Thr Ser Val
225 2
#30 2
#35 2
#40
Arg Lys Leu Ile Leu Phe Leu Glu Thr Thr Le
#u Ser Glu Arg Ala Ser
                                245
# 250
# 255
Arg Gly Ser Phe His Val Ser Gln Ala Ile Le
#u Thr Pro Arg Val Lys
                        260
# 265
# 270
Thr Ile Ala Arg Gly Leu Val Gly Gly Leu Ly
#s Asn Thr Leu Val His
                275
# 280
# 285
Arg Asn Leu Pro Ala Ile Leu Asp Trp Val Ly
#s Thr Gln Lys Pro Gly
        290
# 295
# 300
Ala Met Gly Val Asn Ile Ile Thr Ser Asp Ph
#e Val Asp Leu Val Asp
305 3
#10 3
#15 3
#20
Phe Ala Ala Thr Val Ile Lys Val Glu
                                325
<210> SEQ ID NO 5
<211> LENGTH: 1056
<212> TYPE: DNA
<213> ORGANISM: homo sapiens
<400> SEQUENCE: 5
atgctagcag ttaggaaggc caggaggaaa ctcaggatgg ggaccatctg ct
#cccccaac 60
cccagcggga caaagacatc atcggaggtc tgcaatgccg actggatggc ct
#cgctcccc 120
cctcacctcc acaacctccc cctttccaat ctggcaatcc caggctcaca tg
#attcattc 180
agctactggg tggatgaaaa gtccccagtg gggcctgacc aaacccaagc ta
#tcaaacgc 240
ctcgccagga tctccttggt gaagaagcta atgaagaagt ggtctgtgac tc
#agaacctg 300
acatttcgag aacagctgga agctgggatc cgctactttg acctgcgtgt gt
#cttccaaa 360
ccaggggatg ccgaccagga gatctacttc atccatgggc tttttggcat ca
#aggtctgg 420
gatgggctga tggaaattga ctcgtttctt acacagcacc cccaggagat ta
#tcttcctg 480
gatttcaacc acttctatgc catggatgag acccatcaca aatgcctggt tc
#tgcggatc 540
caggaggcct ttggaaacaa gctgtgccca gcctgcagtg tggaaagttt ga
#cgctgcga 600
actctgtggg agaagaactg ccaggttctt attttctacc actgtccctt ct
#acaagcag 660
taccccttcc tgtggccagg aaagaagatt ccagcgccct gggcaaacac ca
#caagtgtg 720
cgcaaactaa tcctcttctt ggagaccact ctgagtgagc gggcctcacg gg
#gctccttc 780
catgtctccc aagcgatcct cacccccaga gtgaagacca ttgcccgggg ct
#tggttggg 840
ggcctcaaga acacgctggt tcatagacgg agtctcactc tgtcacccaa ac
#tggagtgc 900
agctcttggc tcaccgcagc ctcaacctcc caggctcagg tgattacccc tc
#ctcacaga 960
cagggtttca ccatgtttcc caggctgatc tcaaactcct ggattcaagt ga
#tccaccca 1020
cctcagcccc ccaaagtgcc gggattacag gcatga
#
# 1056
<210> SEQ ID NO 6
<211> LENGTH: 351
<212> TYPE: PRT
<213> ORGANISM: homo sapiens
<400> SEQUENCE: 6
Met Leu Ala Val Arg Lys Ala Arg Arg Lys Le
#u Arg Met Gly Thr Ile
  1 5
# 10
# 15
Cys Ser Pro Asn Pro Ser Gly Thr Lys Thr Se
#r Ser Glu Val Cys Asn
                        20
# 25
# 30
Ala Asp Trp Met Ala Ser Leu Pro Pro His Le
#u His Asn Leu Pro Leu
                35
# 40
# 45
Ser Asn Leu Ala Ile Pro Gly Ser His Asp Se
#r Phe Ser Tyr Trp Val
        50
# 55
# 60
Asp Glu Lys Ser Pro Val Gly Pro Asp Gln Th
#r Gln Ala Ile Lys Arg
65
#70
#75
#80
Leu Ala Arg Ile Ser Leu Val Lys Lys Leu Me
#t Lys Lys Trp Ser Val
                                85
# 90
# 95
Thr Gln Asn Leu Thr Phe Arg Glu Gln Leu Gl
#u Ala Gly Ile Arg Tyr
                        100
# 105
# 110
Phe Asp Leu Arg Val Ser Ser Lys Pro Gly As
#p Ala Asp Gln Glu Ile
                115
# 120
# 125
Tyr Phe Ile His Gly Leu Phe Gly Ile Lys Va
#l Trp Asp Gly Leu Met
        130
# 135
# 140
Glu Ile Asp Ser Phe Leu Thr Gln His Pro Gl
#n Glu Ile Ile Phe Leu
145 1
#50 1
#55 1
#60
Asp Phe Asn His Phe Tyr Ala Met Asp Glu Th
#r His His Lys Cys Leu
                                165
# 170
# 175
Val Leu Arg Ile Gln Glu Ala Phe Gly Asn Ly
#s Leu Cys Pro Ala Cys
                        180
# 185
# 190
Ser Val Glu Ser Leu Thr Leu Arg Thr Leu Tr
#p Glu Lys Asn Cys Gln
                195
# 200
# 205
Val Leu Ile Phe Tyr His Cys Pro Phe Tyr Ly
#s Gln Tyr Pro Phe Leu
        210
# 215
# 220
Trp Pro Gly Lys Lys Ile Pro Ala Pro Trp Al
#a Asn Thr Thr Ser Val
225 2
#30 2
#35 2
#40
Arg Lys Leu Ile Leu Phe Leu Glu Thr Thr Le
#u Ser Glu Arg Ala Ser
                                245
# 250
# 255
Arg Gly Ser Phe His Val Ser Gln Ala Ile Le
#u Thr Pro Arg Val Lys
                        260
# 265
# 270
Thr Ile Ala Arg Gly Leu Val Gly Gly Leu Ly
#s Asn Thr Leu Val His
                275
# 280
# 285
Arg Arg Ser Leu Thr Leu Ser Pro Lys Leu Gl
#u Cys Ser Ser Trp Leu
        290
# 295
# 300
Thr Ala Ala Ser Thr Ser Gln Ala Gln Val Il
#e Thr Pro Pro His Arg
305 3
#10 3
#15 3
#20
Gln Gly Phe Thr Met Phe Pro Arg Leu Ile Se
#r Asn Ser Trp Ile Gln
                                325
# 330
# 335
Val Ile His Pro Pro Gln Pro Pro Lys Val Pr
#o Gly Leu Gln Ala
                        340
# 345
# 350
<210> SEQ ID NO 7
<211> LENGTH: 450
<212> TYPE: DNA
<213> ORGANISM: homo sapiens
<400> SEQUENCE: 7
atgaagaagt ggtctgtgac tcagaacctg acatttcgag aacagctgga ag
#ctgggatc 60
cgctactttg acctgcgtgt gtcttccaaa ccaggggatg ccgaccagga ga
#tctacttc 120
atccatgggc tttttggcat caaggtctgg gatgggctga tggaaattga ct
#cgtttctt 180
acacagcacc cccaggagat tatcttcctg gatttcaacc acttctatgc ca
#tggatgag 240
acccatcaca aatgcctggt tctgcggatc caggaggcct ttggaaacaa gc
#tgtgccca 300
gcctgcagtg tggaaagttt gacgctgcga actctgtggg agaagaactg cc
#aggtagga 360
gagataaact tccaagagca agaatttaac tcttctgctt ttcctgtatt gc
#cggctgta 420
aaatcactca atccagggct cttaggctaa
#
# 450
<210> SEQ ID NO 8
<211> LENGTH: 149
<212> TYPE: PRT
<213> ORGANISM: homo sapiens
<400> SEQUENCE: 8
Met Lys Lys Trp Ser Val Thr Gln Asn Leu Th
#r Phe Arg Glu Gln Leu
  1 5
# 10
# 15
Glu Ala Gly Ile Arg Tyr Phe Asp Leu Arg Va
#l Ser Ser Lys Pro Gly
                        20
# 25
# 30
Asp Ala Asp Gln Glu Ile Tyr Phe Ile His Gl
#y Leu Phe Gly Ile Lys
                35
# 40
# 45
Val Trp Asp Gly Leu Met Glu Ile Asp Ser Ph
#e Leu Thr Gln His Pro
        50
# 55
# 60
Gln Glu Ile Ile Phe Leu Asp Phe Asn His Ph
#e Tyr Ala Met Asp Glu
65
#70
#75
#80
Thr His His Lys Cys Leu Val Leu Arg Ile Gl
#n Glu Ala Phe Gly Asn
                                85
# 90
# 95
Lys Leu Cys Pro Ala Cys Ser Val Glu Ser Le
#u Thr Leu Arg Thr Leu
                        100
# 105
# 110
Trp Glu Lys Asn Cys Gln Val Gly Glu Ile As
#n Phe Gln Glu Gln Glu
                115
# 120
# 125
Phe Asn Ser Ser Ala Phe Pro Val Leu Pro Al
#a Val Lys Ser Leu Asn
        130
# 135
# 140
Pro Gly Leu Leu Gly
145
<210> SEQ ID NO 9
<211> LENGTH: 720
<212> TYPE: DNA
<213> ORGANISM: homo sapiens
<400> SEQUENCE: 9
atgaagaagt ggtctgtgac tcagaacctg acatttcgag aacagctgga ag
#ctgggatc 60
cgctactttg acctgcgtgt gtcttccaaa ccaggggatg ccgaccagga ga
#tctacttc 120
atccatgggc tttttggcat caaggtctgg gatgggctga tggaaattga ct
#cgtttctt 180
acacagcacc cccaggagat tatcttcctg gatttcaacc acttctatgc ca
#tggatgag 240
acccatcaca aatgcctggt tctgcggatc caggaggcct ttggaaacaa gc
#tgtgccca 300
gcctgcagtg tggaaagttt gacgctgcga actctgtggg agaagaactg cc
#aggttctt 360
attttctacc actgtccctt ctacaagcag taccccttcc tgtggccagg aa
#agaagatt 420
ccagcgccct gggcaaacac cacaagtgtg cgcaaactaa tcctcttctt gg
#agaccact 480
ctgagtgagc gggcctcacg gggctccttc catgtctccc aagcgatcct ca
#cccccaga 540
gtgaagacca ttgcccgggg cttggttggg ggcctcaaga acacgctggt tc
#ataggaat 600
cttcctgcca tcctggactg ggtgaaaact cagaagcctg gagccatggg tg
#tcaacatc 660
atcacatctg acttcgtgga cctggtggac tttgctgcga ctgtcatcaa ag
#ttgaatga 720
<210> SEQ ID NO 10
<211> LENGTH: 239
<212> TYPE: PRT
<213> ORGANISM: homo sapiens
<400> SEQUENCE: 10
Met Lys Lys Trp Ser Val Thr Gln Asn Leu Th
#r Phe Arg Glu Gln Leu
  1 5
# 10
# 15
Glu Ala Gly Ile Arg Tyr Phe Asp Leu Arg Va
#l Ser Ser Lys Pro Gly
                        20
# 25
# 30
Asp Ala Asp Gln Glu Ile Tyr Phe Ile His Gl
#y Leu Phe Gly Ile Lys
                35
# 40
# 45
Val Trp Asp Gly Leu Met Glu Ile Asp Ser Ph
#e Leu Thr Gln His Pro
        50
# 55
# 60
Gln Glu Ile Ile Phe Leu Asp Phe Asn His Ph
#e Tyr Ala Met Asp Glu
65
#70
#75
#80
Thr His His Lys Cys Leu Val Leu Arg Ile Gl
#n Glu Ala Phe Gly Asn
                                85
# 90
# 95
Lys Leu Cys Pro Ala Cys Ser Val Glu Ser Le
#u Thr Leu Arg Thr Leu
                        100
# 105
# 110
Trp Glu Lys Asn Cys Gln Val Leu Ile Phe Ty
#r His Cys Pro Phe Tyr
                115
# 120
# 125
Lys Gln Tyr Pro Phe Leu Trp Pro Gly Lys Ly
#s Ile Pro Ala Pro Trp
        130
# 135
# 140
Ala Asn Thr Thr Ser Val Arg Lys Leu Ile Le
#u Phe Leu Glu Thr Thr
145 1
#50 1
#55 1
#60
Leu Ser Glu Arg Ala Ser Arg Gly Ser Phe Hi
#s Val Ser Gln Ala Ile
                                165
# 170
# 175
Leu Thr Pro Arg Val Lys Thr Ile Ala Arg Gl
#y Leu Val Gly Gly Leu
                        180
# 185
# 190
Lys Asn Thr Leu Val His Arg Asn Leu Pro Al
#a Ile Leu Asp Trp Val
                195
# 200
# 205
Lys Thr Gln Lys Pro Gly Ala Met Gly Val As
#n Ile Ile Thr Ser Asp
        210
# 215
# 220
Phe Val Asp Leu Val Asp Phe Ala Ala Thr Va
#l Ile Lys Val Glu
225 2
#30 2
#35
<210> SEQ ID NO 11
<211> LENGTH: 786
<212> TYPE: DNA
<213> ORGANISM: homo sapiens
<400> SEQUENCE: 11
atgaagaagt ggtctgtgac tcagaacctg acatttcgag aacagctgga ag
#ctgggatc 60
cgctactttg acctgcgtgt gtcttccaaa ccaggggatg ccgaccagga ga
#tctacttc 120
atccatgggc tttttggcat caaggtctgg gatgggctga tggaaattga ct
#cgtttctt 180
acacagcacc cccaggagat tatcttcctg gatttcaacc acttctatgc ca
#tggatgag 240
acccatcaca aatgcctggt tctgcggatc caggaggcct ttggaaacaa gc
#tgtgccca 300
gcctgcagtg tggaaagttt gacgctgcga actctgtggg agaagaactg cc
#aggttctt 360
attttctacc actgtccctt ctacaagcag taccccttcc tgtggccagg aa
#agaagatt 420
ccagcgccct gggcaaacac cacaagtgtg cgcaaactaa tcctcttctt gg
#agaccact 480
ctgagtgagc gggcctcacg gggctccttc catgtctccc aagcgatcct ca
#cccccaga 540
gtgaagacca ttgcccgggg cttggttggg ggcctcaaga acacgctggt tc
#atagacgg 600
agtctcactc tgtcacccaa actggagtgc agctcttggc tcaccgcagc ct
#caacctcc 660
caggctcagg tgattacccc tcctcacaga cagggtttca ccatgtttcc ca
#ggctgatc 720
tcaaactcct ggattcaagt gatccaccca cctcagcccc ccaaagtgcc gg
#gattacag 780
gcatga
#
#
# 786
<210> SEQ ID NO 12
<211> LENGTH: 261
<212> TYPE: PRT
<213> ORGANISM: homo sapiens
<400> SEQUENCE: 12
Met Lys Lys Trp Ser Val Thr Gln Asn Leu Th
#r Phe Arg Glu Gln Leu
  1 5
# 10
# 15
Glu Ala Gly Ile Arg Tyr Phe Asp Leu Arg Va
#l Ser Ser Lys Pro Gly
                        20
# 25
# 30
Asp Ala Asp Gln Glu Ile Tyr Phe Ile His Gl
#y Leu Phe Gly Ile Lys
                35
# 40
# 45
Val Trp Asp Gly Leu Met Glu Ile Asp Ser Ph
#e Leu Thr Gln His Pro
        50
# 55
# 60
Gln Glu Ile Ile Phe Leu Asp Phe Asn His Ph
#e Tyr Ala Met Asp Glu
65
#70
#75
#80
Thr His His Lys Cys Leu Val Leu Arg Ile Gl
#n Glu Ala Phe Gly Asn
                                85
# 90
# 95
Lys Leu Cys Pro Ala Cys Ser Val Glu Ser Le
#u Thr Leu Arg Thr Leu
                        100
# 105
# 110
Trp Glu Lys Asn Cys Gln Val Leu Ile Phe Ty
#r His Cys Pro Phe Tyr
                115
# 120
# 125
Lys Gln Tyr Pro Phe Leu Trp Pro Gly Lys Ly
#s Ile Pro Ala Pro Trp
        130
# 135
# 140
Ala Asn Thr Thr Ser Val Arg Lys Leu Ile Le
#u Phe Leu Glu Thr Thr
145 1
#50 1
#55 1
#60
Leu Ser Glu Arg Ala Ser Arg Gly Ser Phe Hi
#s Val Ser Gln Ala Ile
                                165
# 170
# 175
Leu Thr Pro Arg Val Lys Thr Ile Ala Arg Gl
#y Leu Val Gly Gly Leu
                        180
# 185
# 190
Lys Asn Thr Leu Val His Arg Arg Ser Leu Th
#r Leu Ser Pro Lys Leu
                195
# 200
# 205
Glu Cys Ser Ser Trp Leu Thr Ala Ala Ser Th
#r Ser Gln Ala Gln Val
        210
# 215
# 220
Ile Thr Pro Pro His Arg Gln Gly Phe Thr Me
#t Phe Pro Arg Leu Ile
225 2
#30 2
#35 2
#40
Ser Asn Ser Trp Ile Gln Val Ile His Pro Pr
#o Gln Pro Pro Lys Val
                                245
# 250
# 255
Pro Gly Leu Gln Ala
                        260
<210> SEQ ID NO 13
<211> LENGTH: 1426
<212> TYPE: DNA
<213> ORGANISM: homo sapiens
<400> SEQUENCE: 13
attcgcgccc gtcaggcatg tctgtacact gggtgggcac ctgtcttgtg ag
#tggctccg 60
ggtgtggctg ctcctcggac tttcagttta tgtaagattt atctctaggg gc
#ctaccttc 120
ccccatctcc agaggggaac ataagaagtt taacggagct gggactgagc ag
#attaaggg 180
agtggagcgg aggctgggcc ggagagagtg gggactgtga gtgctagtgg gt
#aaggatcc 240
atctgtttgc cccgtccccc agccagaaag gcattttgga aagactggcg tg
#gcaagcgt 300
cgccctgaaa cgtccacaga gcccaagaag tgatgatcac tgagtgagtg gc
#actgggct 360
gagactggcc agtttgttaa caacagggat gctagcagtt aggaaggcca gg
#aggaaact 420
caggatgggg accatctgct cccccaaccc cagcgggaca aagacatcat cg
#gaggtctg 480
caatgccgac tggatggcct cgctcccccc tcacctccac aacctccccc tt
#tccaatct 540
ggcaatccca ggctcacatg attcattcag ctactgggtg gatgaaaagt cc
#ccagtggg 600
gcctgaccaa acccaagcta tcaaacgcct cgccaggatc tccttggtga ag
#aagctaat 660
gaagaagtgg tctgtgactc agaacctgac atttcgagaa cagctggaag ct
#gggatccg 720
ctactttgac ctgcgtgtgt cttccaaacc aggggatgcc gaccaggaga tc
#tacttcat 780
ccatgggctt tttggcatca aggtctggga tgggctgatg gaaattgact cg
#tttcttac 840
acagcacccc caggagatta tcttcctgga tttcaaccac ttctatgcca tg
#gatgagac 900
ccatcacaaa tgcctggttc tgcggatcca ggaggccttt ggaaacaagc tg
#tgcccagc 960
ctgcagtgtg gaaagtttga cgctgcgaac tctgtgggag aagaactgcc ag
#gttcttat 1020
tttctaccac tgtcccttct acaagcagta ccccttcctg tggccaggaa ag
#aagattcc 1080
agcgccctgg gcaaacacca caagtgtgcg caaactaatc ctcttcttgg ag
#accactct 1140
gagtgagcgg gcctcacggg gctccttcca tgtctcccaa gcgatcctca cc
#cccagagt 1200
gaagaccatt gcccggggct tggttggggg cctcaagaac acgctggttc at
#aggaatct 1260
tcctgccatc ctggactggg tgaaaactca gaagcctgga gccatgggtg tc
#aacatcat 1320
cacatctgac ttcgtggacc tggtggactt tgctgcgact gtcatcaaag tt
#gaatgacc 1380
ttctacagga ggacacaagc tctggcttaa tgctgattta attttt
# 1426
<210> SEQ ID NO 14
<211> LENGTH: 2289
<212> TYPE: DNA
<213> ORGANISM: homo sapiens
<400> SEQUENCE: 14
atggcgtccc tgctgcaaga ccagctgacc actgatcagg acttgctgct ga
#tgcaggaa 60
ggcatgccga tgcgcaaggt gaggtccaaa agctggaaga agctaagata ct
#tcagactt 120
cagaatgacg gcatgacagt ctggcatgca cggcaggcca ggggcagtgc ca
#agcccagc 180
ttctcaatct ctgatgtgga gacaatacgt aatggccatg attccgagtt gc
#tgcgtagc 240
ctggcagagg agctccccct ggagcagggc ttcaccattg tcttccatgg cc
#gccgctcc 300
aacctggacc tgatggccaa cagtgttgag gaggcccaga tatggatgcg ag
#ggctccag 360
ctgttggtgg atcttgtcac cagcatggac catcaggagc gcctggacca at
#ggctgagc 420
gattggtttc aacgtggaga caaaaatcag gatggtaaga tgagtttcca ag
#aagttcag 480
cggttattgc acctaatgaa tgtggaaatg gaccaagaat atgccttcag tc
#tttttcag 540
gcagcagaca cgtcccagtc tggaaccctg gaaggagaag aattcgtaca gt
#tctataag 600
gcattgacta aacgtgctga ggtgcaggaa ctgtttgaaa gtttttcagc tg
#atgggcag 660
aagctgactc tgctggaatt tttggatttc ctccaagagg agcagaagga ga
#gagactgc 720
acctctgagc ttgctctgga actcattgac cgctatgaac cttcagacag tg
#gcaaactg 780
cggcatgtgc tgagtatgga tggcttcctc agctacctct gctctaagga tg
#gagacatc 840
ttcaacccag cctgcctccc catctatcag gatatgactc aacccctgaa cc
#actacttc 900
atctgctctt ctcataacac ctacctagtg ggggaccagc tttgcggcca ga
#gcagcgtc 960
gagggatata tacgggccct gaagcggggg tgccgctgcg tggaggtgga tg
#tatgggat 1020
ggacctagcg gggaacctgt cgtttaccac ggacacaccc tgacctcccg ca
#tcctgttc 1080
aaagatgtcg tggccacagt agcacagtat gccttccaga catcagacta cc
#cagtcatc 1140
ttgtccctgg agacccactg cagctgggag cagcagcaga ccatggcccg tc
#atctgact 1200
gagatcctgg gggagcagct gctgagcacc accttggatg gggtgctgcc ca
#ctcagctg 1260
ccctcgcctg aggagcttcg gaggaagatc ctggtgaagg ggaagaagtt aa
#cacttgag 1320
gaagacctgg aatatgagga agaggaagca gaacctgagt tggaagagtc ag
#aattggcg 1380
ctggagtccc agtttgagac tgagcctgag ccccaggagc agaaccttca ga
#ataaggac 1440
aaaaagaaga aatccaagcc catcttgtgt ccagccctct cttccctggt ta
#tctacttg 1500
aagtctgtct cattccgcag cttcacacat tcaaaggagc actaccactt ct
#acgagata 1560
tcatctttct ctgaaaccaa ggccaagcgc ctcatcaagg aggctggcaa tg
#agtttgtg 1620
cagcacaata cttggcagtt aagccgtgtg tatcccagcg gcctgaggac ag
#actcttcc 1680
aactacaacc cccaggaact ctggaatgca ggctgccaga tggtggccat ga
#atatgcag 1740
actgcagggc ttgaaatgga catctgtgat gggcatttcc gccagaatgg cg
#gctgtggc 1800
tatgtgctga agccagactt cctgcgtgat atccagagtt ctttccaccc tg
#agaagccc 1860
atcagccctt tcaaagccca gactctctta atccaggtga tcagcggtca gc
#aactcccc 1920
aaagtggaca agaccaaaga ggggtccatt gtggatccac tggtgaaagt gc
#agatcttt 1980
ggcgttcgtc tagacacagc acggcaggag accaactatg tggagaacaa tg
#gttttaat 2040
ccatactggg ggcagacact atgtttccgg gtgctggtgc ctgaacttgc ca
#tgctgcgt 2100
tttgtggtaa tggattatga ctggaaatcc cgaaatgact ttattggtca gt
#acaccctg 2160
ccttggacct gcatgcaaca aggttaccgc cacattcacc tgctgtccaa ag
#atggcatc 2220
agcctccgcc cagcttccat ctttgtgtat atctgcatcc aggaaggcct gg
#agggggat 2280
gagtcctga
#
#
# 2289
<210> SEQ ID NO 15
<211> LENGTH: 762
<212> TYPE: PRT
<213> ORGANISM: homo sapiens
<400> SEQUENCE: 15
Met Ala Ser Leu Leu Gln Asp Gln Leu Thr Th
#r Asp Gln Asp Leu Leu
  1 5
# 10
# 15
Leu Met Gln Glu Gly Met Pro Met Arg Lys Va
#l Arg Ser Lys Ser Trp
                        20
# 25
# 30
Lys Lys Leu Arg Tyr Phe Arg Leu Gln Asn As
#p Gly Met Thr Val Trp
                35
# 40
# 45
His Ala Arg Gln Ala Arg Gly Ser Ala Lys Pr
#o Ser Phe Ser Ile Ser
        50
# 55
# 60
Asp Val Glu Thr Ile Arg Asn Gly His Asp Se
#r Glu Leu Leu Arg Ser
65
#70
#75
#80
Leu Ala Glu Glu Leu Pro Leu Glu Gln Gly Ph
#e Thr Ile Val Phe His
                                85
# 90
# 95
Gly Arg Arg Ser Asn Leu Asp Leu Met Ala As
#n Ser Val Glu Glu Ala
                        100
# 105
# 110
Gln Ile Trp Met Arg Gly Leu Gln Leu Leu Va
#l Asp Leu Val Thr Ser
                115
# 120
# 125
Met Asp His Gln Glu Arg Leu Asp Gln Trp Le
#u Ser Asp Trp Phe Gln
        130
# 135
# 140
Arg Gly Asp Lys Asn Gln Asp Gly Lys Met Se
#r Phe Gln Glu Val Gln
145 1
#50 1
#55 1
#60
Arg Leu Leu His Leu Met Asn Val Glu Met As
#p Gln Glu Tyr Ala Phe
                                165
# 170
# 175
Ser Leu Phe Gln Ala Ala Asp Thr Ser Gln Se
#r Gly Thr Leu Glu Gly
                        180
# 185
# 190
Glu Glu Phe Val Gln Phe Tyr Lys Ala Leu Th
#r Lys Arg Ala Glu Val
                195
# 200
# 205
Gln Glu Leu Phe Glu Ser Phe Ser Ala Asp Gl
#y Gln Lys Leu Thr Leu
        210
# 215
# 220
Leu Glu Phe Leu Asp Phe Leu Gln Glu Glu Gl
#n Lys Glu Arg Asp Cys
225 2
#30 2
#35 2
#40
Thr Ser Glu Leu Ala Leu Glu Leu Ile Asp Ar
#g Tyr Glu Pro Ser Asp
                                245
# 250
# 255
Ser Gly Lys Leu Arg His Val Leu Ser Met As
#p Gly Phe Leu Ser Tyr
                        260
# 265
# 270
Leu Cys Ser Lys Asp Gly Asp Ile Phe Asn Pr
#o Ala Cys Leu Pro Ile
                275
# 280
# 285
Tyr Gln Asp Met Thr Gln Pro Leu Asn His Ty
#r Phe Ile Cys Ser Ser
        290
# 295
# 300
His Asn Thr Tyr Leu Val Gly Asp Gln Leu Cy
#s Gly Gln Ser Ser Val
305 3
#10 3
#15 3
#20
Glu Gly Tyr Ile Arg Ala Leu Lys Arg Gly Cy
#s Arg Cys Val Glu Val
                                325
# 330
# 335
Asp Val Trp Asp Gly Pro Ser Gly Glu Pro Va
#l Val Tyr His Gly His
                        340
# 345
# 350
Thr Leu Thr Ser Arg Ile Leu Phe Lys Asp Va
#l Val Ala Thr Val Ala
                355
# 360
# 365
Gln Tyr Ala Phe Gln Thr Ser Asp Tyr Pro Va
#l Ile Leu Ser Leu Glu
        370
# 375
# 380
Thr His Cys Ser Trp Glu Gln Gln Gln Thr Me
#t Ala Arg His Leu Thr
385 3
#90 3
#95 4
#00
Glu Ile Leu Gly Glu Gln Leu Leu Ser Thr Th
#r Leu Asp Gly Val Leu
                                405
# 410
# 415
Pro Thr Gln Leu Pro Ser Pro Glu Glu Leu Ar
#g Arg Lys Ile Leu Val
                        420
# 425
# 430
Lys Gly Lys Lys Leu Thr Leu Glu Glu Asp Le
#u Glu Tyr Glu Glu Glu
                435
# 440
# 445
Glu Ala Glu Pro Glu Leu Glu Glu Ser Glu Le
#u Ala Leu Glu Ser Gln
        450
# 455
# 460
Phe Glu Thr Glu Pro Glu Pro Gln Glu Gln As
#n Leu Gln Asn Lys Asp
465 4
#70 4
#75 4
#80
Lys Lys Lys Lys Ser Lys Pro Ile Leu Cys Pr
#o Ala Leu Ser Ser Leu
                                485
# 490
# 495
Val Ile Tyr Leu Lys Ser Val Ser Phe Arg Se
#r Phe Thr His Ser Lys
                        500
# 505
# 510
Glu His Tyr His Phe Tyr Glu Ile Ser Ser Ph
#e Ser Glu Thr Lys Ala
                515
# 520
# 525
Lys Arg Leu Ile Lys Glu Ala Gly Asn Glu Ph
#e Val Gln His Asn Thr
        530
# 535
# 540
Trp Gln Leu Ser Arg Val Tyr Pro Ser Gly Le
#u Arg Thr Asp Ser Ser
545 5
#50 5
#55 5
#60
Asn Tyr Asn Pro Gln Glu Leu Trp Asn Ala Gl
#y Cys Gln Met Val Ala
                                565
# 570
# 575
Met Asn Met Gln Thr Ala Gly Leu Glu Met As
#p Ile Cys Asp Gly His
                        580
# 585
# 590
Phe Arg Gln Asn Gly Gly Cys Gly Tyr Val Le
#u Lys Pro Asp Phe Leu
                595
# 600
# 605
Arg Asp Ile Gln Ser Ser Phe His Pro Glu Ly
#s Pro Ile Ser Pro Phe
        610
# 615
# 620
Lys Ala Gln Thr Leu Leu Ile Gln Val Ile Se
#r Gly Gln Gln Leu Pro
625 6
#30 6
#35 6
#40
Lys Val Asp Lys Thr Lys Glu Gly Ser Ile Va
#l Asp Pro Leu Val Lys
                                645
# 650
# 655
Val Gln Ile Phe Gly Val Arg Leu Asp Thr Al
#a Arg Gln Glu Thr Asn
                        660
# 665
# 670
Tyr Val Glu Asn Asn Gly Phe Asn Pro Tyr Tr
#p Gly Gln Thr Leu Cys
                675
# 680
# 685
Phe Arg Val Leu Val Pro Glu Leu Ala Met Le
#u Arg Phe Val Val Met
        690
# 695
# 700
Asp Tyr Asp Trp Lys Ser Arg Asn Asp Phe Il
#e Gly Gln Tyr Thr Leu
705 7
#10 7
#15 7
#20
Pro Trp Thr Cys Met Gln Gln Gly Tyr Arg Hi
#s Ile His Leu Leu Ser
                                725
# 730
# 735
Lys Asp Gly Ile Ser Leu Arg Pro Ala Ser Il
#e Phe Val Tyr Ile Cys
                        740
# 745
# 750
Ile Gln Glu Gly Leu Glu Gly Asp Glu Ser
                755
# 760
<210> SEQ ID NO 16
<211> LENGTH: 210
<212> TYPE: DNA
<213> ORGANISM: homo sapiens
<400> SEQUENCE: 16
atggcgtccc tgctgcaaga ccagctgacc actgatcagg acttgctgct ga
#tgcaggaa 60
ggcatgccga tgcgcaagtc tcaatctctg atgtggagac aatacgtaat gg
#ccatgatt 120
ccgagttgct gcgtagcctg gcagaggagc tccccctgga gcagggcttc ac
#cattgtct 180
tccatggccg ccgctccaac ctggacctga
#
# 210
<210> SEQ ID NO 17
<211> LENGTH: 69
<212> TYPE: PRT
<213> ORGANISM: homo sapiens
<400> SEQUENCE: 17
Met Ala Ser Leu Leu Gln Asp Gln Leu Thr Th
#r Asp Gln Asp Leu Leu
  1 5
# 10
# 15
Leu Met Gln Glu Gly Met Pro Met Arg Lys Se
#r Gln Ser Leu Met Trp
                        20
# 25
# 30
Arg Gln Tyr Val Met Ala Met Ile Pro Ser Cy
#s Cys Val Ala Trp Gln
                35
# 40
# 45
Arg Ser Ser Pro Trp Ser Arg Ala Ser Pro Le
#u Ser Ser Met Ala Ala
        50
# 55
# 60
Ala Pro Thr Trp Thr
65
<210> SEQ ID NO 18
<211> LENGTH: 819
<212> TYPE: DNA
<213> ORGANISM: homo sapiens
<400> SEQUENCE: 18
atggcgtccc tgctgcaaga ccagctgacc actgatcagg acttgctgct ga
#tgcaggaa 60
ggcatgccga tgcgcaaggt gaggtccaaa agctggaaga agctaagata ct
#tcagactt 120
cagaatgacg gcatgacagt ctggcatgca cggcaggcca ggggcagtgc ca
#agcccagc 180
ttctcaatct ctgatgtgga gacaatacgt aatggccatg attccgagtt gc
#tgcgtagc 240
ctggcagagg agctccccct ggagcagggc ttcaccattg tcttccatgg cc
#gccgctcc 300
aacctggacc tgatggccaa cagtgttgag gaggcccaga tatggatgcg ag
#ggctccag 360
ctgttggtgg atcttgtcac cagcatggac catcaggagc gcctggacca at
#ggctgagc 420
gattggtttc aacgtggaga caaaaatcag gatggtaaga tgagtttcca ag
#aagttcag 480
cggttattgc acctaatgaa tgtggaaatg gaccaagaat atgccttcag tc
#tttttcag 540
gcagcagaca cgtcccagtc tggaaccctg gaaggagaag aattcgtaca gt
#tctataag 600
gcattgacta aacgtgctga ggtgcaggaa ctgtttgaaa gtttttcagc tg
#atgggcag 660
aagctgactc tgctggaatt tttggatttc ctccaagagg agcagaagga ga
#gagactgc 720
acctctgagc ttgctctgga actcattgac cgctatgaac cttcagacag tg
#gagcttcg 780
gaggaagatc ctggtgaagg ggaagaagtt aacacttga
#
# 819
<210> SEQ ID NO 19
<211> LENGTH: 272
<212> TYPE: PRT
<213> ORGANISM: homo sapiens
<400> SEQUENCE: 19
Met Ala Ser Leu Leu Gln Asp Gln Leu Thr Th
#r Asp Gln Asp Leu Leu
  1 5
# 10
# 15
Leu Met Gln Glu Gly Met Pro Met Arg Lys Va
#l Arg Ser Lys Ser Trp
                        20
# 25
# 30
Lys Lys Leu Arg Tyr Phe Arg Leu Gln Asn As
#p Gly Met Thr Val Trp
                35
# 40
# 45
His Ala Arg Gln Ala Arg Gly Ser Ala Lys Pr
#o Ser Phe Ser Ile Ser
        50
# 55
# 60
Asp Val Glu Thr Ile Arg Asn Gly His Asp Se
#r Glu Leu Leu Arg Ser
65
#70
#75
#80
Leu Ala Glu Glu Leu Pro Leu Glu Gln Gly Ph
#e Thr Ile Val Phe His
                                85
# 90
# 95
Gly Arg Arg Ser Asn Leu Asp Leu Met Ala As
#n Ser Val Glu Glu Ala
                        100
# 105
# 110
Gln Ile Trp Met Arg Gly Leu Gln Leu Leu Va
#l Asp Leu Val Thr Ser
                115
# 120
# 125
Met Asp His Gln Glu Arg Leu Asp Gln Trp Le
#u Ser Asp Trp Phe Gln
        130
# 135
# 140
Arg Gly Asp Lys Asn Gln Asp Gly Lys Met Se
#r Phe Gln Glu Val Gln
145 1
#50 1
#55 1
#60
Arg Leu Leu His Leu Met Asn Val Glu Met As
#p Gln Glu Tyr Ala Phe
                                165
# 170
# 175
Ser Leu Phe Gln Ala Ala Asp Thr Ser Gln Se
#r Gly Thr Leu Glu Gly
                        180
# 185
# 190
Glu Glu Phe Val Gln Phe Tyr Lys Ala Leu Th
#r Lys Arg Ala Glu Val
                195
# 200
# 205
Gln Glu Leu Phe Glu Ser Phe Ser Ala Asp Gl
#y Gln Lys Leu Thr Leu
        210
# 215
# 220
Leu Glu Phe Leu Asp Phe Leu Gln Glu Glu Gl
#n Lys Glu Arg Asp Cys
225 2
#30 2
#35 2
#40
Thr Ser Glu Leu Ala Leu Glu Leu Ile Asp Ar
#g Tyr Glu Pro Ser Asp
                                245
# 250
# 255
Ser Gly Ala Ser Glu Glu Asp Pro Gly Glu Gl
#y Glu Glu Val Asn Thr
                        260
# 265
# 270
<210> SEQ ID NO 20
<211> LENGTH: 2709
<212> TYPE: DNA
<213> ORGANISM: homo sapiens
<400> SEQUENCE: 20
aagagctcac acctttcccc ttcttactgc ttccctccgg ctataacttg cc
#agtcacag 60
cagccagctg ctgtagaaga ggggaggaaa caagccagtg caaggggagc aa
#aagagaaa 120
aggagccagg ctgggcttcc tgatcccaca gcatcgcaga gctcgggagg ca
#cagctcac 180
agacacagga aacacaggac tgctattctg ctctcctgcc cacggtgatc tg
#gtgccagc 240
tggtggaaca gtgggtgatg gcgtccctgc tgcaagacca gctgaccact ga
#tcaggact 300
tgctgctgat gcaggaaggc atgccgatgc gcaaggtgag gtccaaaagc tg
#gaagaagc 360
taagatactt cagacttcag aatgacggca tgacagtctg gcatgcacgg ca
#ggccaggg 420
gcagtgccaa gcccagcttc tcaatctctg atgtggagac aatacgtaat gg
#ccatgatt 480
ccgagttgct gcgtagcctg gcagaggagc tccccctgga gcagggcttc ac
#cattgtct 540
tccatggccg ccgctccaac ctggacctga tggccaacag tgttgaggag gc
#ccagatat 600
ggatgcgagg gctccagctg ttggtggatc ttgtcaccag catggaccat ca
#ggagcgcc 660
tggaccaatg gctgagcgat tggtttcaac gtggagacaa aaatcaggat gg
#taagatga 720
gtttccaaga agttcagcgg ttattgcacc taatgaatgt ggaaatggac ca
#agaatatg 780
ccttcagtct ttttcaggca gcagacacgt cccagtctgg aaccctggaa gg
#agaagaat 840
tcgtacagtt ctataaggca ttgactaaac gtgctgaggt gcaggaactg tt
#tgaaagtt 900
tttcagctga tgggcagaag ctgactctgc tggaattttt ggatttcctc ca
#agaggagc 960
agaaggagag agactgcacc tctgagcttg ctctggaact cattgaccgc ta
#tgaacctt 1020
cagacagtgg caaactgcgg catgtgctga gtatggatgg cttcctcagc ta
#cctctgct 1080
ctaaggatgg agacatcttc aacccagcct gcctccccat ctatcaggat at
#gactcaac 1140
ccctgaacca ctacttcatc tgctcttctc ataacaccta cctagtgggg ga
#ccagcttt 1200
gcggccagag cagcgtcgag ggatatatac gggccctgaa gcgggggtgc cg
#ctgcgtgg 1260
aggtggatgt atgggatgga cctagcgggg aacctgtcgt ttaccacgga ca
#caccctga 1320
cctcccgcat cctgttcaaa gatgtcgtgg ccacagtagc acagtatgcc tt
#ccagacat 1380
cagactaccc agtcatcttg tccctggaga cccactgcag ctgggagcag ca
#gcagacca 1440
tggcccgtca tctgactgag atcctggggg agcagctgct gagcaccacc tt
#ggatgggg 1500
tgctgcccac tcagctgccc tcgcctgagg agcttcggag gaagatcctg gt
#gaagggga 1560
agaagttaac acttgaggaa gacctggaat atgaggaaga ggaagcagaa cc
#tgagttgg 1620
aagagtcaga attggcgctg gagtcccagt ttgagactga gcctgagccc ca
#ggagcaga 1680
accttcagaa taaggacaaa aagaagaaat ccaagcccat cttgtgtcca gc
#cctctctt 1740
ccctggttat ctacttgaag tctgtctcat tccgcagctt cacacattca aa
#ggagcact 1800
accacttcta cgagatatca tctttctctg aaaccaaggc caagcgcctc at
#caaggagg 1860
ctggcaatga gtttgtgcag cacaatactt ggcagttaag ccgtgtgtat cc
#cagcggcc 1920
tgaggacaga ctcttccaac tacaaccccc aggaactctg gaatgcaggc tg
#ccagatgg 1980
tggccatgaa tatgcagact gcagggcttg aaatggacat ctgtgatggg ca
#tttccgcc 2040
agaatggcgg ctgtggctat gtgctgaagc cagacttcct gcgtgatatc ca
#gagttctt 2100
tccaccctga gaagcccatc agccctttca aagcccagac tctcttaatc ca
#ggtgatca 2160
gcggtcagca actccccaaa gtggacaaga ccaaagaggg gtccattgtg ga
#tccactgg 2220
tgaaagtgca gatctttggc gttcgtctag acacagcacg gcaggagacc aa
#ctatgtgg 2280
agaacaatgg ttttaatcca tactgggggc agacactatg tttccgggtg ct
#ggtgcctg 2340
aacttgccat gctgcgtttt gtggtaatgg attatgactg gaaatcccga aa
#tgacttta 2400
ttggtcagta caccctgcct tggacctgca tgcaacaagg ttaccgccac at
#tcacctgc 2460
tgtccaaaga tggcatcagc ctccgcccag cttccatctt tgtgtatatc tg
#catccagg 2520
aaggcctgga gggggatgag tcctgaggtg ggcatttcac gggaagggtt gg
#tatgctgg 2580
ctttagacgg ggagaaacat ctggaaggat gctcgagaga acaaatggag gt
#ggtgaaaa 2640
tcaagctttg gattgtgcat tcctaggcac aaaattacct cattcttcct aa
#caagcaat 2700
ctgggacct
#
#
# 2709
```

1. An isolated nucleic acid molecule comprising at least 24 contiguous bases of nucleotide sequence first disclosed in SEQ ID NO:1.

2. An isolated nucleic acid molecule comprising a nucleotide sequence that:
(a) encodes the amino acid sequence shown in SEQ ID NO:2; and
(b) hybridizes under stringent conditions to the nucleotide sequence of SEQ ID NO:1 or the complement thereof.

3. An isolated nucleic acid molecule encoding the amino acid sequence described in SEQ ID NO:2.

4. An isolated oligopeptide comprising at least about 12 amino acids in a sequence first disclosed in SEQ ID NO:2.

5. An isolated nucleic acid molecule encoding the amino acid sequence described in SEQ ID NO:4.

6. An isolated nucleic acid molecule encoding the amino acid sequence described in SEQ ID NO:6.

7. An isolated nucleic acid molecule encoding the amino acid sequence described in SEQ ID NO:12.

8. An isolated nucleic acid molecule comprising at least 24 contiguous bases of nucleotide sequence first disclosed in SEQ ID NO:14.

9. An isolated nucleic acid molecule comprising a nucleotide sequence that:
(a) encodes the amino acid sequence shown in SEQ ID NO:15; and
(b) hybridizes under stringent conditions to the nucleotide sequence of SEQ ID NO:14 or the complement thereof.

10. An isolated nucleic acid molecule encoding the amino acid sequence described in SEQ ID NO:15.

11. An isolated oligopeptide comprising at least about 12 amino acids in a sequence first disclosed in SEQ ID NO:15.

12. An isolated nucleic acid molecule encoding the amino acid sequence described in SEQ ID NO:17.

13. An isolated nucleic acid molecule encoding the amino acid sequence described in SEQ ID NO:19.
